(19) United States
(12) Patent Application Publication
(10) Pub. No.: US 2026/0337163 A1
(43) Pub. Date: Oct. 29, 2026

(54) A Method for the Construction of Aminocyclobutanes from Copper-Catalyzed Aqueous [2+2] Cycloadditions of Unactivated Olefins

(71) Applicant: The Board of Trustees of the Leland Stanford Junior University, Stanford, CA (US)

(72) Inventors: Noah Burns, Stanford, CA (US); Carol Mansson, Stanford, CA (US)

(21) Appl. No.: 19/101,033

(22) PCT Filed: Jul. 31, 2023
(86) PCT No.: PCT/US2023/029087

§ 371 Date: Feb. 4, 2025

Related U.S. Application Data
(60) Provisional application No. 63/395,683, filed on Aug. 5, 2022.
(60) Provisional application No. 63/417,851, filed on Oct. 20, 2022.

(51) Int. Cl. C07D 209/52

(52) U.S. Cl. CPC C07D 209/52

(57) ABSTRACT

Provided are methods of producing a cyclobutane compound. In some embodiments, the methods include contacting an un-activated diene with a copper(II) catalyst under conditions so that the un-activated diene undergoes a [2±2]cycloaddition to produce the cyclobutane compound.

### CROSS-REFERENCE TO RELATED APPLICATION

Pursuant to 35 U.S.C. § 119 (e), this application claims priority to the filing date of U.S. Provisional Patent Application Ser. No. 63/395,683 filed Aug. 5, 2022, and U.S. Provisional Patent Application Ser. No. 63/417,851 filed Oct. 20, 2022, the disclosures of which applications are herein incorporated by reference.

### GOVERNMENT RIGHTS

This invention was made with Government support under contract N00014-18-1-2659 awarded by the Office of Naval Research. The Government has certain rights in the invention.

### INTRODUCTION

In the world of medicinal chemistry, cyclobutanes offer valuable degrees of structural tuning as rigid and three-dimensional pharmacophores and as a means to “escape from flatland,” a noteworthy pursuit in modern pharmaceutical chemistry.^{1,2′3,4} Their installation is becoming increasingly utilized for lead generation, with photochemical [2+2] reactions standing as arguably the most efficient way to make four-membered rings, cyclizing relatively simple olefin precursors with full atom economy and the potential to set four contiguous stereocenters.⁵ Although a well-established reaction, the vast majority of [2+2] photocycloadditions rely on activated olefins, where at least one of the reacting partners has extended τ conjugation (FIG. 1A).⁶ Currently, a method for the photochemical [2+2]cycloaddition between two un-activated olefins is the Kochi-Salomon reaction, wherein two alkyl-substituted olefins can be cyclized with catalytic CuOTf upon irradiation with UV light (FIG. 1B).^{7,8,9} A remarkable transformation, it has been largely unexplored since its development in the 1970s and suffers from a number of drawbacks. The Cu(I) catalyst is highly air- and moisture-sensitive; the reaction scope is primarily limited to standard oxygen functionality and sterically bulky olefins tend to be sluggish or non-reactive. Recently, Yoon and coworkers disclosed a substantially more active Cu(I) catalyst with a hexafluoroantimonate (SbF₆⁻) counteranion and 1,5-cyclooctadiene as a stabilizing ligand.¹⁰ This resulted in increased reaction rates and even allowed for the formation of highly substituted cyclobutanes from more substituted alkenes. Still, there are remaining drawbacks in the use of moisture- and air-sensitive catalysts and the limited functional group tolerance which excludes certain common Lewis or Brønsted basic functionality, most notably amines and amides which are ubiquitous in biologically active small molecules. Despite the fact that amines pose methodological challenges associated with their low oxidation potential, Lewis basicity, and high nucleophilic character, we recognized that an amine-tolerant Kochi-Salomon reaction would allow rapid access to aminocyclobutanes (FIG. 1C). Such compounds are desirable pharmacological motifs, in particular the 3-azabicyclo[3.2.0]heptane scaffold which can serve as a bioisostere for pyrrolidines and piperidines.¹¹

### REFERENCES

(1) Lovering, F.; Bikker, J.; Humblet, C. J. Med. Chem. 2009, 52, 6752
(2) Marson, C. M. Chem. Soc. Rev. 2011, 40, 5514
(3) van der Kolk, M. R.; Janssen, M. A. C. H., Rutjes, F. P. J. T., Blanco-Ania D. MedChemMed 2022, 17, e202200020
(4) Carreira, E. M.; Fressard, T. C. Chem. Rev. 2014, 114, 8257
(5) Cox, B.; Booker-Milburn, K. I.; Elliott, L. D.; Robertson-Ralph, M.; Zdorichenko, V. ACS Med. Chem. Lett. 2019, 10, 1512
(6) Poplata, S.; Tröster, A.; Zou, Y.-Q.; Bach, T. Chem. Rev. 2016, 116, 9748
(7) Salomon, R. G.; Kochi, J. K. J. Am. Chem. Soc. 1974, 96, 1137
(8) Salomon, R. G.; Folting, K.; Streib, W. E.; Kochi, J. K. J. Am. Chem. Soc. 1974, 96, 1145
(9) Ghosh, S.; Raychaudhuri, S. R.; Salomon, R. G. J. Org. Chem. 1987, 52, 83
(10) Gravatt, C. S.; Melecio-Zambrano L.; Yoon, T. P. Angew. Chem. Int. Ed. 2021, 60, 3989
(11) Denisenko, A. V.; Druzhenko, T.; Skalenko, Y.; Samoilenko, M.; Grygorenko, O. O.; Zozulya, S.; Mykhailiuk, P. K. J. Org. Chem. 2017, 82, 9627

### SUMMARY

Provided are methods of producing a cyclobutane compound. In some embodiments, the methods include contacting an un-activated diene with a copper(II) catalyst under conditions so that the un-activated diene undergoes a [2+2]cycloaddition to produce the cyclobutane compound.

### BRIEF DESCRIPTION OF THE FIGURES

FIG. 1A shows the activated olefin requirement of nearly all photochemical [2+2]cycloadditions.

FIG. 1B shows the Kochi-Salomon reaction and its drawbacks.

FIG. 1C shows representative examples of pharmacological scaffolds containing aminobicyclo[3.2.0]heptanes.

FIG. 1D shows a new, practical, amine-tolerant reaction system for cyclization of un-activated olefins.

FIG. 1E shows the discovery and optimization of amine-tolerant Kochi-Salomon reaction. Yields were determined by internal standard (DMSO) (a) or isolated as BF₃ adduct (b).

FIG. 2 shows substrate scope of amine-tolerant Kochi-Salomon reaction. ^{a} AcOH used in place of H₂SO₄; ^{b} Isolated as HCL salt; ^{c} 10 hr reaction time; ^{d} 0.05 M reaction concentration; ^{e} 48 hours reaction time.

FIG. 3 shows derivatization of amine-tolerant Kochi-Salomon reaction products. Panel (a) shows general derivatization to more complex scaffolds. Panel (b) shows pharmacologically relevant derivatization to known and novel drug analogues.

FIG. 4 shows the effect of different catalysts, acids, and reaction conditions on Pdt:SM ratios.

### DEFINITIONS

The terms “un-activated diene” and “un-activated diene” are used interchangeably herein.

“Alkyl” refers to a monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases the alkyl group has 1 to 24 carbon atoms, e.g. 1 to 12, 1 to 6, or 1 to 3.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl.

“Alkynyl” refers to a monoradical, branched or linear, non-cyclic hydrocarbonyl group that comprises a carbon-carbon triple bond. Exemplary alkynyl groups include ethynyl and n-propynyl.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g. O, S, N) as a ring atom and that is not aromatic (i.e. distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e. none of the ring atoms are heteroatoms (e.g. O, S, N). In some cases the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the aromatic ring is a heteroatom (e.g. O, S, N). Exemplary heteroaryl groups include those obtained from removing a hydrogen atom from pyridine, pyrimidine, furan, thiophene, or benzothiophene.

The term “substituted” refers the removal of one or more hydrogens from an atom (e.g. from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group.

As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no long be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

Exemplary substituents include alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some cases, the substitutions can themselves be further substituted with one or more groups. For example, the group —C₆H₄CH₂CH₃ can be considered as substituted aryl, i.e. an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein. In some cases, the substituents are not substituted with any other groups.

Diradical groups are also described herein, i.e. in contrast to the monoradical groups such as alkyl and aryl described above. The term “alkylene” refers to the diradical version of an alkyl group, i.e. an alkylene group is a diradical, branched or linear, cyclic or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e. a CHCH₃ fragment where the first atom has two single bonds to other two different groups). The term “arylene” refers to the diradical version of an aryl group, e.g. 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Acyl” refers to a group of formula —C(O)R wherein R is alkyl, alkenyl, alkynyl, or substituted versions thereof. For example, the acetyl group has formula —C(O)CH₃. “Carbonyl” refers to a diradical group of formula —C(O)—.

“Alkoxy” refers to a group of formula —O(alkyl). Similar groups can be derived from alkenyl, alkynyl, aryl, heteroaryl, and other groups.

“Amino” refers to the group —NR^{X}R^{Y} wherein R^{X} and R^{Y} are each independently H or a non-hydrogen substituent. Exemplary non-hydrogen substituents include alkyl groups (e.g. methyl, ethyl, and isopropyl).

“Carbonyl” refers to a diradical group of formula —C(O)—.

“Carboxy” is used interchangeably with carboxyl and carboxylate to refer to the —CO₂H group and salts thereof.

“Ether” refers to a diradical group of formula —O—. For instance, if the ether group is connected to an alkyl group, then the overall group is an alkoxy group (e.g. —OCH₃ or methoxy). If the ether is connected to a carbonyl group, then the overall group is an ester group of formula —OC(O)—.

“Halo” and “halogen” refer to the chloro, bromo, fluoro, and iodo groups.

“Nitro” refers to the group of formula —NO₂.

Unless otherwise specified, reference to an atom is meant to include all isotopes of that atom. For example, reference to H includes ¹H, ²H (i.e. D or deuterium) and ³H (i.e. tritium), and reference to C is includes both ¹²C and all other isotopes of carbon (e.g. ¹³C). Unless specified otherwise, groups include all possible stereoisomers.

### DETAILED DESCRIPTION

Provided are methods of producing a cyclobutane compound. In some embodiments, the methods include contacting an un-activated diene with a copper(II) catalyst under conditions so that the un-activated diene undergoes a [2+2]cycloaddition to produce the cyclobutane compound.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, provided are methods of producing a cyclobutane compound. As used herein, the term “cyclobutane compound” refers to a compound comprising a cyclobutane group. In some cases, the method includes the step of contacting an un-activated diene with a copper(II) catalyst under conditions sufficient so that the un-activated diene undergoes a [2+2]cycloaddition to produce the cyclobutane compound.

In some embodiments, the contacting is performed in an aqueous solvent. Stated in another manner, the un-activated diene, copper(II) catalysts, and other optional components are present in a solution comprising water during the contacting step. For example, 95% or more of the mass of the solution can be water, such as 97% or more, 99% or more, or 99.5% or more.

In some cases, the contacting is performed in a non-aqueous solution. In some cases the non-aqueous solution comprises a non-aqueous solvent (e.g., diethyl ether, which is also written as “Et₂O”) and a significant amount of water (e.g., 1% or more by mass).

In some cases, contacting conditions include irradiating with light, e.g., wherein the light have a wavelength ranging from 200 nm to 400 nm, such as from 220 nm to 350 nm, from 210 nm to 300 nm, or from 220 nm to 260 nm. In some cases, the spectrum of the irradiation light has a maximum value at a wavelength ranging from 200 nm to 400 nm, such as from 220 to 350 nm, or from 220 to 360 nm. Stated in another manner, the irradiation can be performed with light emitted by a light source that has an emission maximum at a wavelength ranging from 200 nm to 400 nm, such as from 220 to 350 nm, or from 220 to 360 nm. In some cases, the light source is a laser. For example, He—Ag⁺ lasers can produce 224 nm light, KrF excimer light sources can produce 248 nm light, and XeCl excimer light sources can produce 308 nm light.

In some embodiments, the copper(II) catalyst is selected from the group consisting of CuSO₄, Cu(OTf)₂, Cu(OAc)₂, CuCl₂, and hydrates thereof. In some cases, the catalyst is present at a concentration ranging from 1 mol % to 50 mol %, such as 5 mol % to 20 mol %. As used herein, a 5 mol % catalyst loading means that the number of moles of catalyst divided by the number of moles of un-activated diene is 0.05, i.e., 5%.

In some cases, the contacting occurs in the presence of an acid, e.g., a Brønsted acid. In some embodiments, the Brønsted acid is selected from the group consisting of sulfuric acid, tetrafluoroboric acid (HBF₄), triflic acid (TfOH), trifluoroacetic acid (TFA), acetic acid (AcOH), and hydrochloric acid (HCl). In some cases, the acid is present in a mol % of 80% to 120% relative to the un-activated diene, e.g., from 90% to 110%. Stated in another manner, the acid can be present at a concentration of 80 mol % to 120 mol %, such as from 90 mol % to 110 mol %.

In some cases, the un-activated diene has formula (IA):

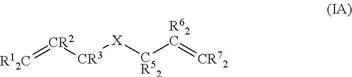

wherein:
X is NR⁴, CR⁴₂, or (CR⁴)₂; and
each R¹-R⁷ is independently selected from the group consisting of H, alkyl, substituted alkyl, cycloalkyl, substituted cycloalkyl, aryl, substituted aryl, heteroaryl, substituted heteroaryl, and amino, provided that if X is (CR⁴)₂ then two R⁴ groups on adjacent carbon atoms can, along with the atoms to which they are attached, form a cyclic ring; and
the cyclobutane compound has the formula (IB):

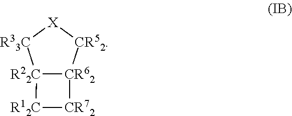

In some cases, each R¹, R², R⁶, and R⁷ is independently selected from the group consisting of H, alkyl, and substituted alkyl. In some embodiments, each R¹, R², R⁶, and R⁷ is H. In some cases, each R³ and R⁵ is H. In some cases, X is NR⁴, e.g., wherein R⁴ is selected from the group consisting of alkyl, substituted alkyl, aryl, and substituted aryl.

In some cases, the un-activated diene is an aminodiene, e.g., where one or more of X or a substituent R¹-R⁷ includes an amino group.

In some cases, the method is capable of producing the cyclobutane group even though the un-activated has other chemical functionalities, i.e., the method has good functional group tolerance. In some embodiments, the un-activated diene comprises an amine group, an amide group, a cyclic ring, a carboxylic acid group, or a combination thereof.

In some cases, each R¹, R², R³, R⁵, R⁶, and R⁷ is independently selected from the group consisting of H, alkyl, substituted alkyl, cycloalkyl. Such groups do not have τ-electrons at the alpha position, and therefore the C═C double bonds drawn in formula (IA) are not τ-conjugated to any r-electrons of the R¹, R², R³, R⁵, R⁶, or R⁷ groups.

In some cases, the method is capable of producing the cyclobutane group despite the presence of oxygen (i.e., O₂). In some cases, the contacting is performed in the presence of oxygen, e.g., the contacting is performed in a solvent (e.g., an aqueous solvent) comprising dissolved oxygen. The maximum concentration of dissolved oxygen in water at 20° C. was reported to be about 9 mg per liter by the U.S. Environmental Protection Agency (archive.epa.gov/water/archive/web/html/vms52.html). Thus, in some cases the contacting is performed when the amount of dissolved oxygen is 0.5 mg/L or more, such as 1 mg/L or more or 5 mg/L or more.

The chemical reactions can be performed at relatively ambient temperatures, i.e., without requiring significant heating. For example, the reaction can be performed at a temperature 90° C. or less, such as 80° C. or less, 70° C. or less, 60° C. or less, 50° C. or less, 40° C. or less, or 25° C. or less. In some cases, the reaction is performed at a temperature ranging from 5° C. to 30° C. In some cases, the contacting is performed for 48 hours or less, such as 24 hours or less, 12 hours or less, 6 hours or less, 3 hours or less, or 1 hour or less. In some embodiments, 50% or more of the un-activated diene is converted to the cyclobutane compound, such as 75% or more, 90% or more, or 95% or more. In some embodiments, the contacting is performed at a temperature ranging from 10° C. to 30° C. for 24 hours or less and 25% or more of the un-activated diene is converted to the cyclobutane compound, such as 50% or more or 75% or more.

### Aqueous Mixtures

Aspects of the invention also include an aqueous mixture for producing a cyclobutane compound, wherein mixture comprising: an un-activated diene, a copper(II) catalyst, and an aqueous solvent.

In some embodiments, the copper(II) catalyst is selected from the group consisting of CuSO₄, Cu(OTf)₂, Cu(OAc)₂, CuCl₂, and hydrates thereof. In some cases, the catalyst is present at a concentration ranging from 1 mol % to 50 mol %, such as 5 mol % to 20 mol %.

In some cases, the contacting occurs in the presence of an acid, e.g., a Brønsted acid. In some embodiments, the Brønsted acid is selected from the group consisting of sulfuric acid, tetrafluoroboric acid (HBF₄), triflic acid (TfOH), trifluoroacetic acid (TFA), acetic acid (AcOH), and hydrochloric acid (HCl). In some cases, the acid is present in a mol % of 80% to 120% relative to the un-activated diene, e.g., from 90% to 110%.

In some cases, the un-activated diene has formula (IA):

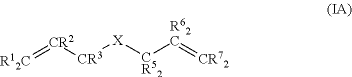

wherein:
X is NR⁴, CR⁴₂, or (CR⁴)₂; and
each R¹-R⁷ is independently selected from the group consisting of H, alkyl, substituted alkyl, cycloalkyl, substituted cycloalkyl, aryl, substituted aryl, heteroaryl, substituted heteroaryl, and amino, provided that if X is (CR⁴)₂ then two R⁴ groups on adjacent carbon atoms can, along with the atoms to which they are attached, form a cyclic ring; and the cyclobutane compound has the formula (IB):

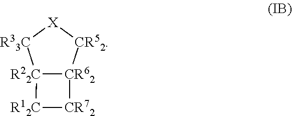

In some cases, each R¹, R², R⁶, and R⁷ is independently selected from the group consisting of H, alkyl, and substituted alkyl. In some embodiments, each R¹, R², R⁶, and R⁷ is H. In some cases, each R³ and R⁵ is H. In some cases, X is NR⁴, e.g., wherein R⁴ is selected from the group consisting of alkyl, substituted alkyl, aryl, and substituted aryl.

In some cases, the un-activated diene is an aminodiene, e.g., where one or more of X or a substituent R¹-R⁷ includes an amino group.

In some cases, the method is capable of producing the cyclobutane group even though the un-activated has other chemical functionalities, i.e., the method has good functional group tolerance. In some embodiments, the un-activated diene comprises an amine group, an amide group, a cyclic ring, a carboxylic acid group, an N⁺—O⁻ group, or a combination thereof.

In some cases, the method is capable of producing the cyclobutane group despite the presence of oxygen (i.e., O₂), e.g., wherein the aqueous solvent comprises dissolved oxygen. For example, in some cases the amount of dissolved oxygen is 0.5 mg/L or more, such as 1 mg/L or more or 5 mg/L or more.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1

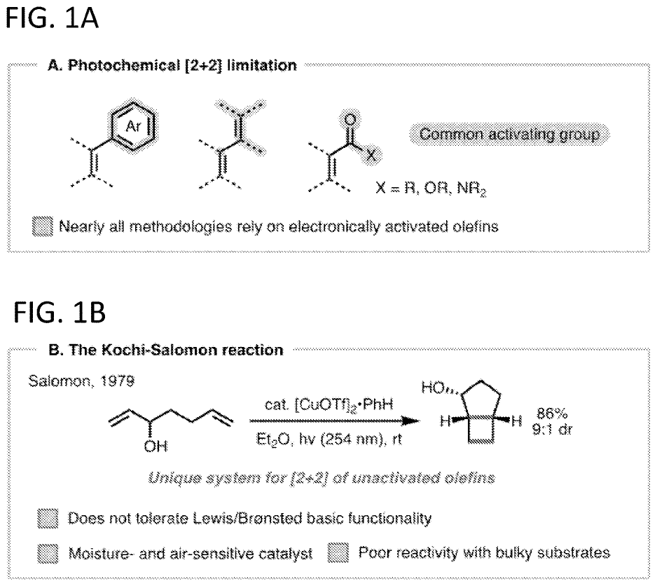

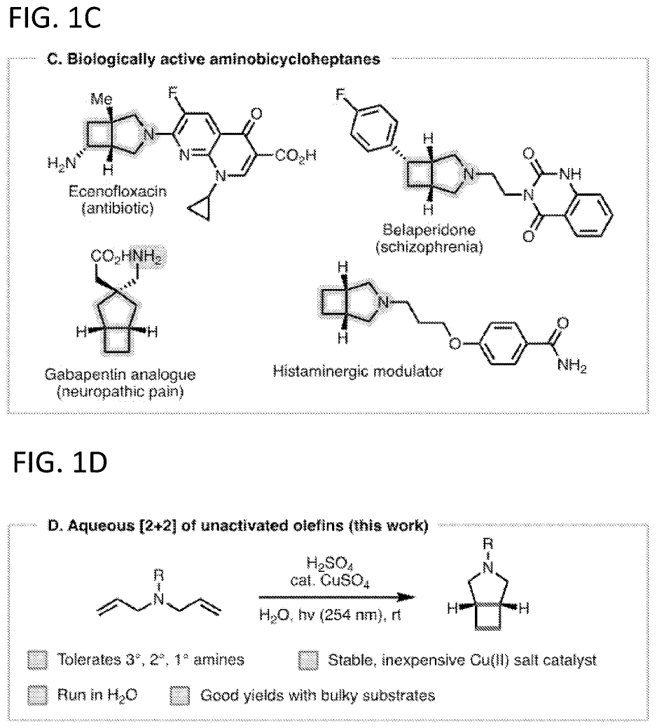

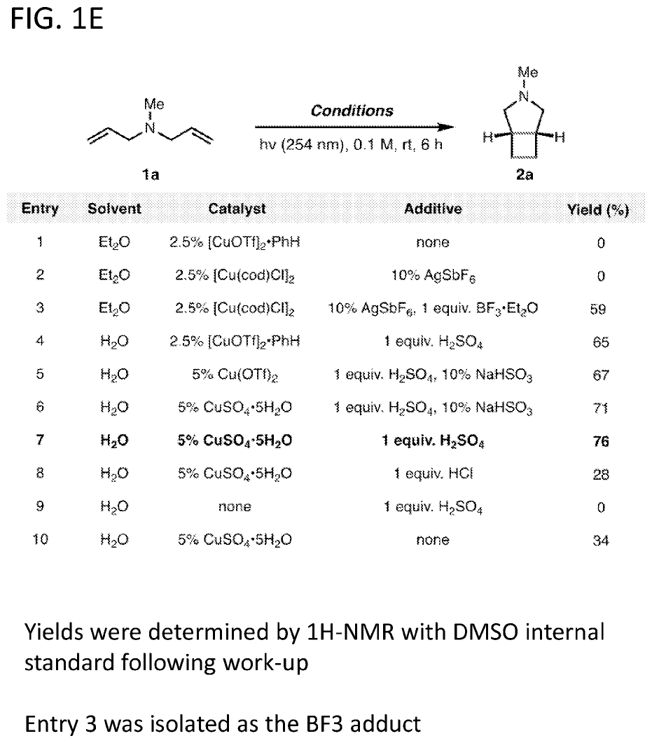

In an effort to address the problems described above, remarkably simple method was developed for the synthesis of aminocyclobutanes from their corresponding un-activated diene precursors (FIG. 1D). This approach provides a practical, scalable, and green method for the [2+2]cycloaddition of unprotected amine-containing un-activated dienes using some of the simplest and most widely available reagents in any chemistry lab. Furthermore, a variety of product derivatizations was demonstrated as well as the synthesis of both known and novel pharmacological analogues.

Development of this amine tolerant [2+2]cycloaddition was begun by confirming that the standard conditions developed by Kochi and Salomon did not produce any cyclobutane product 2a from diallylmethylamine 1a (FIG. 1E, entry 1), nor did the current conditions developed by Yoon and coworkers (entry 2). Based on previous success in other areas,^{12,13,14,15} it was anticipated that a Lewis or Brønsted acid could mask the amines in situ. It was observed that addition of the Lewis acid BF₃·Et₂O afforded exclusively the cyclized product as its BF₃ adduct (entry 3). While providing a solid proof-of-concept, this approach had features that could be improved. Firstly, some amine substrates appeared to be rendered completely insoluble as the BF₃ adduct, hindering any reactivity. Secondly, if cyclization occurred, the release of the free amine from the adduct proved surprisingly difficult, requiring prolonged treatment with concentrated base. In this regard, it was recognized that masking the amine with a simpler Brønsted acid would represent an attractive way of attaining the [2+2] products, as it could be easily removed in a standard work-up. However, there was concern regarding the insolubility of ammonium salts in Et₂O, the preferred solvent for the Kochi-Salomon reaction. Water, the natural solvent choice for solubility, seemed unlikely as it is understood that these [2+2]cycloadditions require the intermediacy of a highly electrophilic Cu(I) center which would likely be destroyed in an aqueous environment.^{7,16} Although Cu(OTf)₂ is precedented for the standard Kochi-Salomon reaction in dry organic solvents, this is likely due to an in-situ reduction to an active Cu(1) species.¹⁷ However, it was hypothesized that the addition of a reducing agent might generate enough active catalyst to provide the desired product. Using [CuOTf]2·PhH and H₂SO₄ gave 67% yield (entry 4). It was observed that the reaction proceeded smoothly in water with Cu(OTf)₂ in the presence of NaHSO₃, using H₂SO₄ as the Brønsted acid (entry 5). The free amine could simply be obtained with a basic work-up. Next, an objective was to simplify and optimize the reaction conditions. It was found that CuSO₄·5H₂O gave ideal reactivity and surprisingly, no reducing agent was required (entry 7). These optimal conditions are notable as CuSO₄·5H₂O is the most inexpensive Cu(II) salt and H₂SO₄ is one of the most common mineral acids. Switching the acid to HCl gave a slower reaction (entry 8). Omitting the catalyst gave no product (entry 9). Omitting the acid did provide some product (entry 10), and it is possible that this is due to partial protective protonation in the neutral aqueous environment. However, substrate decomposition prevented higher product yields and substrate solubility worsened with decreasing reactant polarity speaking to the effect of added acid.

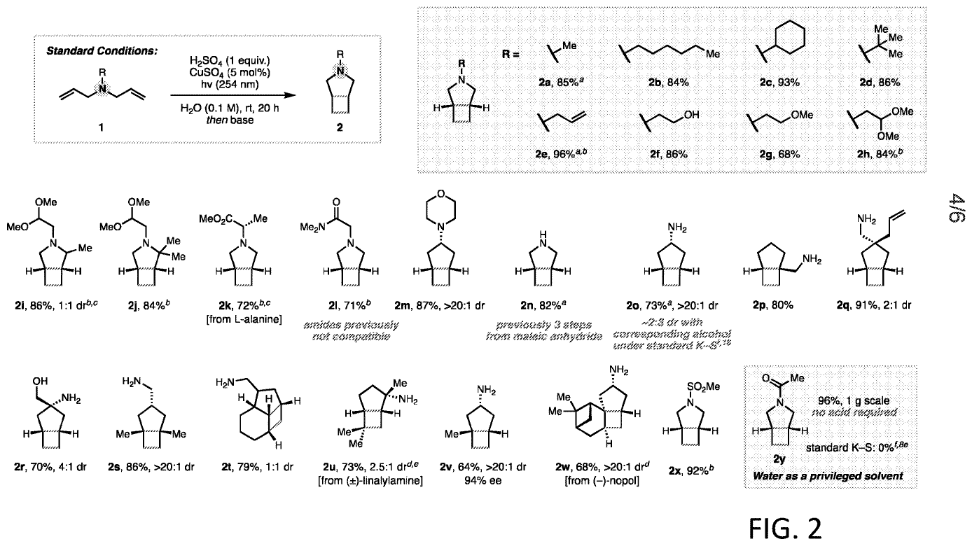

Next, the reaction scope was explored (FIG. 2), starting with substituted diallylamines. Me, 1°, 2° and 3° alkyl diallylamines gave cyclized products in high yield (2a-2d). A third allyl group did not interfere with the reaction (2e). Ethers and free alcohols were also tolerated (2f, 2g). Switching the acid to milder AcOH permitted the [2+2]cycloaddition of acid sensitive substrates like dimethyl acetals (2h), including ones with both mono- and di-substitution at the allylic position (2i, 2j). The reaction also proceeded smoothly with esters to give cyclobutanes such as 2k, derived from amino acid L-alanine. Amides are functional groups previously not tolerated in any reported Cu(I) catalyzed [2+2]cycloaddition, yet it was found that that amide substituted diallylamine cyclized in good yield (2l) under the current conditions. While allylamines represent a fine class of substrates, the amine can be present at any position like in a morpholine heterocycle giving 2m in high yield with very good diastereoselectivity. Moreover, unprotected 2° and 1° amines serve as excellent candidates for this cyclization (2n-2w). Notably, 3-azabicyclo[3.2.0]heptane (2n) is directly accessed from diallylamine, a significant improvement over the previous three-step procedure hampered by an activated [2+2]cycloaddition using ethylene gas.¹⁸ The amine substrates may also give better diastereoselectivity than their oxygen counterparts as exemplified by the highly selective formation of 20 whereas hepta-1,6-dien-4-ol gives approx. 3:2 dr under standard Kochi-Salomon conditions.¹⁹ Furthermore, it was found that even sterically hindered cyclobutanes could be formed in good yields including cyclobutanes containing vicinal quaternary centers (2s), various fused cyclobutanes derived from cyclic olefins (2t, 2w) and the cyclized product of linalylamine (2u). Although not a basic amine, N,N-diallylmethanesulfonamide cyclized in high yield to provide 2x. Finally, to probe whether water is an ideal solvent in any other capacity than dissolving ammonium salts, we turned our attention to N,N-diallylacetamide. Finally, to probe whether water is a good solvent in any other capacity than dissolving ammonium salts, attention was turned to diallylacetamide. Under standard Kochi-Salomon conditions, diallylacetamide is known to be completely unreactive,²⁰ however, it was possible to recover cyclized product 2y in excellent yield under the conditions even when acid was omitted, showing that water is indeed a privileged solvent.

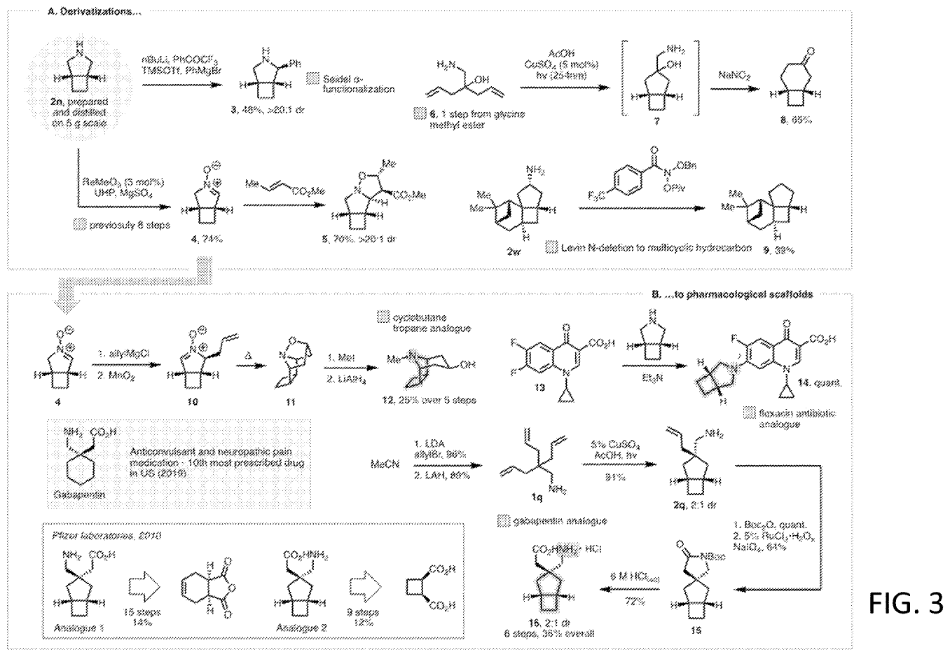

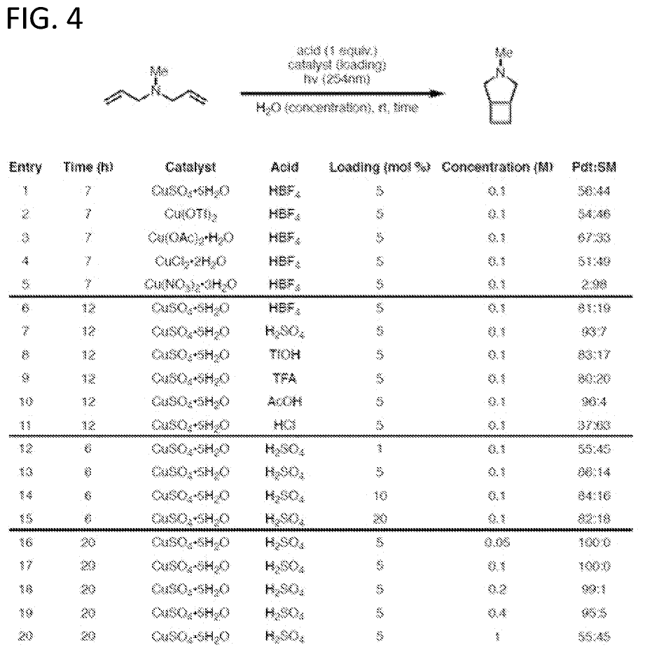

Functionalized pyrrolidines are valuable building blocks in many pharmaceuticals. As a result, its bicyclic analogues 3-azabicyclo[3.1.0]hexane and 3-azabicyclo[3.3.0]octane have been introduced as skeletal surrogates for both pyrrolidines and piperidines. Nevertheless, their middle sibling, 3-azabicyclo[3.2.0]heptane (2n), remains an order of magnitude less explored,¹⁸ presumably due to a lack of efficient synthetic access as suggested by its high price ($980/g).²¹ It was recognized that the current chemistry could provide rapid access to this small, yet rare amine and were able to produce multi-gram quantities at a time. Although the yield is diminished at this large scale, the current method still lends itself well to distilling significant quantities of expensive 2n from inexpensive diallylamine ($0.15/g), CuSO₄·5H₂O ($0.05/g) and H₂SO₄ ($0.03/g) in a single step (FIG. 3, panel A). To demonstrate the synthetic utility of the amine containing cyclobutane products, it was decided to functionalize 2n, applying chemistry developed by Seidel and coworkers²² to install an a-phenyl group to give 3 in excellent diastereoselectivity. Furthermore, 2n was directly converted into nitrone 4, whose synthesis had previously required eight steps from maleic anhydride.²³ 4 could then be engaged in a dipolar [3+2]cycloaddition with methyl crotonate to generate complex tricyclic scaffold 5 with excellent diastereoselectivity. Aminoalcohol 6, which is accessed in one step from a glycine ester, lends itself well to a one-pot [2+2]cycloaddition/Tiffeneau-Demjanov ring expansion to generate 6,4 fused bicycle 8 with a ketone synthetic handle. Drawing upon recent chemistry developed by Levin and coworkers,²⁴ nitrogen was deleted on primary amine 2w to furnish hydrocarbon 9. There was then focus on applying the current chemistry to pharmacologically relevant scaffolds (FIG. 3, panel B). Since numerous medications are based on the tropane skeleton,²⁵ it was thought it apt to synthesize its cyclobutane analogue (12). Notably, this was accomplished from 4 in a five-step sequence that required no purification of intermediates. Fluoroquinolones are a class of antibiotics commonly used against resistant bacteria in case of severe infections. Possessing the same skeleton, they mainly differ in the amine at C7. A new analogue (14) of this class was synthesized through an SNAr between difluoroquinolone 13 and 2n that proceeded in quantitative yield. Finally, it was realized the potential of this chemistry to rapidly assemble bicyclic gabapentin analogues. Gabapentin is one of the most prescribed medications in the US, of which many analogues have been investigated. Among the most interesting compounds, disclosed by Pfizer laboratories, contain the bicyclo[3.2.0]heptane motif (analogues 1 and 2).²⁶ Both were assembled in lengthy sequences in less-than-optimal overall yield. It was envisioned that a significantly shorter path starting with the common laboratory solvent acetonitrile. This route enabled the synthesis of the analogues 16 in six steps and 36% overall yield. The diastereoselectivity is subpar and will be the target of future studies of this reaction.

While the undeniable synthetic power of the [2+2]cycloaddition has led to a flurry of methodologies, practically none of them have been able to break away from the activated olefin requirement. The Kochi-Salomon reaction is therefore a unique well of potential that has remained largely unexplored for almost 50 years. In the current efforts to popularize this [2+2]cycloaddition, the reaction scope has been broadened to include unprotected, basic nitrogens and amides while at the same time designing a reaction run under aqueous conditions with standard mineral acids and Cu(II) salts. It is hoped that the practicality and accessibility of this reaction will encourage its use in the future synthesis of amine-containing cyclobutanes.

In addition, the effect of different reaction conditions on products was tested for a model compound, as shown in FIG. 5. In particular, entries 1-5 correspond to the different copper(II) catalysts, showing that Cu(OAc)₂—H₂O gave the highest Pdt:SM ratio at 67:33. Next, entries 6-11 tested the effect of acid, showing that AcOH gave the highest Pdt:SM ratio. Entries 12-15 tested the effect of copper loading, showing that only 1 mol % gave the lowest Pdt:SM ratio of 55:45, with 5%, 10%, and 20% giving results of about 82:18 to 86:14. Lastly, entries 16-20 tested different concentrations of acid, ranging from 0.05 M sulfuric acid to 1 M sulfuric acid, wherein the 1 M reaction gave 55:45 Pdt:SM whereas the other lower concentrations gave ratios of 95:5 to 100:0.

### REFERENCES

(12) Howell, J. M.; Feng, K.; Clark, J. R.; Trzepkowski, L. J.; White, M. C. J. Am. Chem. Soc. 2015, 137,14590
(13) Lee, M.; Sanford, M. S. J. Am. Chem. Soc. 2015, 137, 12796
(14) Mack, J. B. C.; Gipson, J. D.; Du Bois, J.; Sigman, M. S. J. Am. Chem. Soc. 2017, 139, 9503
(15) Robinson, S. G.; Mack, J. B. C.; Alektiar, S. N.; Du Bois, J.; Sigman, M. S. Org. Lett. 2020, 22, 7060
(16) Jayasekara, G. K.; Antolini, C.; Smith, M. A.; Jacoby, D. J.; Escolastico, J.; Girard, N.; Young, B. T.; Hayes, D. J. Am. Chem. Soc. 2021,
(17) Langer K.; Mattay, J.; Heidbreder, A.; Möller, M. Liebigs Ann. Chem. 1992, 257
(18) Skalenko, Y.; Druzhenko, T.; Denisenko, A. V.; Samoilenko, M.; Dacenko, O. P.; Trofymchuk, S. A.; Grygorenko, O. O.; Tolmachev, A. A.; Mykhailiuk, P. K. J. Org. Chem. 2018, 83, 6275
(19) Evers, J. T. M.; Mackor, A. Tetrahedron Lett. 1978, 19, 821
(20) Salomon, R. G.; Ghosh, S.; Raychaudhuri, S. R.; Miranti, T. S. Tetrahedron Lett. 1984, 25, 3167
(21) Lowest list price of commercial suppliers (MolPort) listed on SciFinder (05/2022)
(22) Paul, A.; Seidel, D. J. Am. Chem. Soc. 2019, 141, 8778
(23) Tufariello, J. J.; Milowsky, A. S.; Al-Nuri, M.; Goldstein, S. Tetrahedron Lett. 1987, 46, 267
(24) Berger, K. J.; Driscoll, J. L.; Yuan, M.; Dherange, B. D.; Gutierrez, O.; Levin, M. D. J. Am. Chem. Soc. 2021, 143, 17366
(25) See examples: Transderm Scop® (i.e., scopolamine), Spiriva® (i.e., tiotropium bromide), Regurin® (i.e., trospium chloride) among others. See review: Kohnen-Johannsen, K. L.; Kayser, O. Molecules 2019, 24, 796
(26) Blakemore, D. C.; Bryans, J. S.; Carnell, P.; Carr, C. L. Chessum, N. E. A.; Field, M. J.; Kinsella, N.; Osborne, S. A.; Warren, A. N.; Williams, S. C. Bioorg. Med. Chem. Lett. 2010, 20, 461

Notwithstanding the appended claims, the disclosure is also defined by the following clauses:
1. A method of producing a cyclobutane compound, the method comprising:
contacting an un-activated diene with a copper(II) catalyst under conditions sufficient so that the un-activated diene undergoes a [2+2]cycloaddition to produce the cyclobutane compound.
2. The method of clause 1, wherein the contacting is performed in an aqueous solvent.
3. The method of any of the preceding clauses, wherein the conditions comprise irradiating with light.
4. The method of clause 3, wherein the light has a wavelength ranging from 200 nm to 400 nm.
5. The method of clause 4, wherein the conditions comprise irradiating with light from a light source having an emission maximum wavelength ranging from 200 nm to 400 nm.
6. The method of any of the preceding clauses, wherein the copper(II) catalyst is selected from the group consisting of CuSO₄, Cu(OTf)₂, Cu(OAc)₂, CuCl₂, and hydrates thereof.
7. The method of any of the preceding clauses, wherein the contacting occurs in the presence of an acid.
8. The method of clause 7, wherein the acid is a Brønsted acid.
9. The method of clause 8, wherein the Brønsted acid is selected from the group consisting of sulfuric acid, tetrafluoroboric acid (HBF₄), triflic acid (TfOH), trifluoroacetic acid (TFA), acetic acid (AcOH), and hydrochloric acid (HCl).
10. The method of any of clauses 7-9, wherein the acid is present in a mol % of 80% to 120% relative to the un-activated diene.
11. The method of any of the preceding clauses, wherein the un-activated diene has formula (IA):

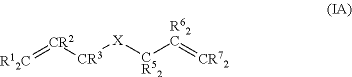

wherein:
   X is NR⁴, CR⁴₂, or (CR⁴)₂; and
   each R¹-R⁷ is independently selected from the group consisting of H, alkyl, substituted alkyl, cycloalkyl, substituted cycloalkyl, aryl, substituted aryl, heteroaryl, substituted heteroaryl, and amino, provided that if X is (CR⁴)₂ then two R⁴ groups on adjacent carbon atoms can, along with the atoms to which they are attached, form a cyclic ring; and
   the cyclobutane compound has the formula (IB):

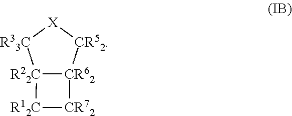

12. The method of clause 11, wherein each R¹, R², R⁶, and R⁷ is independently selected from the group consisting of H, alkyl, and substituted alkyl.
13. The method of any one of clauses 11-12, wherein each R¹, R², R⁶, and R⁷ is H.
14. The method of any one of clauses 11-13, wherein each R³ and R⁵ is H.
15. The method of any one of clauses 11-14, wherein X is NR⁴.
16. The method of clause 15, R⁴ is selected from the group consisting of alkyl, substituted alkyl, aryl, and substituted aryl.
17. The method of any of the preceding clauses, wherein the un-activated diene comprises an amine group.
18. The method of any of the preceding clauses, wherein the un-activated diene comprises an amide group.
19. The method of any of the preceding clauses, wherein the un-activated diene comprises cyclic ring, a carboxylic acid group, an N⁺—O⁻ group, or a combination thereof.
20. The method of any of the preceding clauses, wherein the contacting is performed in the presence of oxygen.
21. The method of clause 20, wherein contacting is performed with a solvent comprising dissolved oxygen at a concentration of 0.5 mg/L or more.
22. The method of any of the preceding clauses, wherein the contacting is performed for 48 hours or less.
23. The method of any of the preceding clauses, wherein the temperature during the contacting is 60° C. or less.
24. The method of any of the preceding clauses, wherein 50% or more of the un-activated diene is converted to the cyclobutane compound during the contacting.
25. An aqueous mixture for producing a cyclobutane compound, the mixture comprising:
   an un-activated diene;
   a copper(II) catalyst; and
   an aqueous solvent.
26. The mixture of clause 25, wherein the copper(II) catalyst is selected from the group consisting of CuSO₄, Cu(OTf)₂, Cu(OAc)₂, CuCl₂, and hydrates thereof.
27. The mixture of any one of clauses 25-26, further comprising an acid.
28. The mixture of clause 27, wherein the acid is a Brønsted acid.
29. The mixture of clause 28, wherein the Brønsted acid is selected from the group consisting of sulfuric acid, tetrafluoroboric acid (HBF₄), triflic acid (TfOH), trifluoroacetic acid (TFA), acetic acid (AcOH), and hydrochloric acid (HCl).
30. The mixture of any one of clauses 27-29, wherein the acid is present in a mol % of 80% to 120% relative to the un-activated diene.
31. The mixture of any one of clauses 25-30, wherein the un-activated diene has formula (IA):

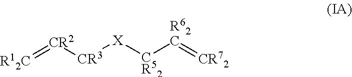

wherein:
   X is NR⁴, CR⁴₂, or (CR⁴)₂; and
   each R¹-R⁷ is independently selected from the group consisting of H, alkyl, substituted alkyl, cycloalkyl, substituted cycloalkyl, aryl, substituted aryl, heteroaryl, substituted heteroaryl, and amino, provided that if X is (CR⁴)₂ then two R⁴ groups on adjacent carbon atoms can, along with the atoms to which they are attached, form a cyclic ring; and
   the cyclobutane compound has the formula (IB):

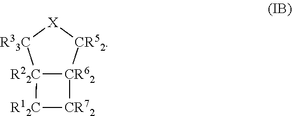

32. The mixture of clause 31, wherein each R¹, R², R⁶, and R⁷ is independently selected from the group consisting of H, alkyl, and substituted alkyl.
33. The mixture of any one of clauses 31-32, wherein each R¹, R², R⁶, and R⁷ is H.
34. The mixture of any one of clauses 31-33, wherein each R³ and R⁵ is H.
35. The mixture of any one of clauses 31-34, wherein X is NR⁴.
36. The mixture of clause 35, R⁴ is selected from the group consisting of alkyl, substituted alkyl, aryl, and substituted aryl.
37. The mixture of any one of clauses 25-36, wherein the un-activated diene comprises an amine group.
38. The mixture of any one of clauses 25-37, wherein the un-activated diene comprises an amide group.
39. The mixture of any one of clauses 25-38, wherein the un-activated diene comprises cyclic ring, a carboxylic acid group, an N⁺—O⁻ group, or a combination thereof.
40. The mixture of any one of clauses 25-39, wherein the aqueous mixture comprises dissolved oxygen.
41. The method of clause 40, wherein the aqueous mixture comprises dissolved oxygen at a concentration of 0.5 mg/L or more.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

1. A method of producing a cyclobutane compound, the method comprising:
contacting an un-activated diene with a copper(II) catalyst under conditions sufficient so that the un-activated diene undergoes a [2+2]cycloaddition to produce the cyclobutane compound.

2. The method of claim 1, wherein the contacting is performed in an aqueous solvent.

3. The method of claim 1, wherein the conditions comprise irradiating with light, such as light having a wavelength ranging from 200 nm to 400 nm.

4. The method of claim 1, wherein the copper(II) catalyst is selected from the group consisting of CuSO₄, Cu(OTf)₂, Cu(OAc)₂, CuCl₂, and hydrates thereof.

5. The method of claim 1, wherein the contacting occurs in the presence of an acid, such as a Brønsted acid.

6. The method of claim 5, wherein the Brønsted acid is selected from the group consisting of sulfuric acid, tetrafluoroboric acid (HBF₄), triflic acid (TfOH), trifluoroacetic acid (TFA), acetic acid (AcOH), and hydrochloric acid (HCl).

7. The method of claim 1, wherein the un-activated diene has formula (IA): 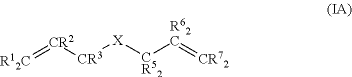
wherein:
X is NR⁴, CR⁴₂, or (CR⁴)₂; and
each R¹-R⁷ is independently selected from the group consisting of H, alkyl, substituted alkyl, cycloalkyl, substituted cycloalkyl, aryl, substituted aryl, heteroaryl, substituted heteroaryl, and amino, provided that if X is (CR⁴)₂ then two R⁴ groups on adjacent carbon atoms can, along with the atoms to which they are attached, form a cyclic ring; and
the cyclobutane compound has the formula (IB):

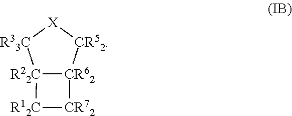

8. The method of claim 7, wherein each R¹, R², R⁶, and R⁷ is independently selected from the group consisting of H, alkyl, and substituted alkyl.

9. The method of claim 7, wherein each R¹, R², R⁶, and R⁷ is H.

10. The method of claim 7, wherein each R³ and R⁵ is H.

11. The method of claim 7, wherein X is NR⁴.

12. The method of claim 11, R⁴ is selected from the group consisting of alkyl, substituted alkyl, aryl, and substituted aryl.

13. The method of claim 7, wherein the un-activated diene comprises an amine group and/or an amide group.

14. The method of claim 7, wherein the un-activated diene comprises cyclic ring, a carboxylic acid group, an N⁺—O⁻ group, or a combination thereof.

15. The method of claim 7, wherein the contacting is performed in the presence of oxygen.

16. An aqueous mixture for producing a cyclobutane compound, the mixture comprising:
an un-activated diene;
a copper(II) catalyst; and
an aqueous solvent.
